(19) United States
(12) Patent Application Publication
(10) Pub. No.: US 2017/0209045 A1
(43) Pub. Date: Jul. 27, 2017

(54) SYSTEM AND METHOD FOR INTRAOCULAR PRESSURE SENSING

(71) Applicants: CALIFORNIA INSTITUTE OF TECHNOLOGY, Pasadena, CA (US); THE REGENTS OF THE UNIVERSITY OF CALIFORNIA, Oakland, CA (US)

(72) Inventors: Hyuck Choo, San Marino, CA (US); Jeongoen Lee, Pasadena, CA (US); David W. Sretavan, Tiburon, CA (US)

(21) Appl. No.: 15/417,128

(22) Filed: Jan. 26, 2017

Related U.S. Application Data
(60) Provisional application No. 62/408,269, filed on Oct. 14, 2016.
(60) Provisional application No. 62/287,307, filed on Jan. 26, 2016.

(51) Int. Cl. A61B 3/16; A61B 5/00

(52) U.S. Cl. CPC A61B 3/16; A61B 5/7271; A61B 5/0075

(57) ABSTRACT

IOP sensor and system for measuring intraocular pressure are disclosed herein. Examples embodiments of the IOP sensor can include: a chamber having a first wall and a second wall opposing the first wall; a first array of photonic components disposed inside of the chamber; a raised portion located within the chamber on a surface of the second wall; at least one side wall separating the first and second walls; and a layer of anti-reflective coating within the chamber.

### CROSS-REFERENCE TO RELATED APPLICATIONS

This application claims the benefit of and priority to U.S. Provisional Application No. 62/287,307, filed on Jan. 26, 2016, and U.S. Provisional Application No. 62/408,269, filed on Jan. 26, 2016, both of which are incorporated herein by reference in their entireties and for all purposes.

### STATEMENT REGARDING FEDERALLY-SPONSORED RESEARCH OR DEVELOPMENT

These inventions were made with government support under Grant No. EY024582 awarded by the National Institutes of Health. The government has certain rights in the inventions.

### FIELD

Various aspects of the disclosure relate to an intraocular pressure sensing system that can include a sensor and a reader.

### BACKGROUND

Glaucoma is a leading cause of blindness, affecting an estimated four million Americans and seventy million individuals globally. As glaucoma typically affects the elderly, the aging demographic trends indicate that this disease will continue to be an ever-increasing socioeconomic burden to society. Elevated intraocular pressure (“IOP”) is a major risk factor for glaucoma, and IOP monitoring is the single most important clinical management tool.

Despite the pervasive use of IOP readings for disease monitoring and the clinically proven importance of the aggressive lowering of IOP, current clinical management is primarily based on only periodic snapshots of IOP in the doctor's office obtained every few months. The inability of patients to easily monitor their own IOPs at different times of the day or during various daily activities hinders the comprehensive understanding of the IOP profile of individual patients and the possibility of custom-tailored IOP control.

The need for better IOP monitoring in clinical ophthalmology and in disease research has been widely appreciated. Existing measurement techniques in clinical use measure IOP indirectly. Current IOP measurements involve a form of contact or noncontact application tonometry. However, both modalities have difficulties in providing reliable and repeatable readouts of actual IOP values inside the eye. All tonometers produce indirect IOP readings by deforming the ocular globe and correlating this deformation to the pressure within the eye. Their readouts are heavily influenced by the corneal curvature and thickness, or corneal mechanical properties that vary due to co-existing ocular pathologies. For example, patients who have received laser photorefractive keratectomy have thinner corneas in the treated eyes and consistently show lower IOP when measured using tonometry techniques.

Tonometry currently requires specialized equipment operated by an ophthalmologist, optometrist, or skilled technician. Hence, IOP measurements are made typically in a doctor's office about two to four times per year. Since studies show that IOP varies widely throughout the day, quarterly measurements are poor representations of a patient's actual IOP profile.

### SUMMARY

Example embodiments of an IOP sensing system disclosed, as are example embodiments of components of the system (sensor, reader, etc.) and methods of using the system and/or components thereof and methods of manufacturing the system and/or components thereof. Certain embodiments of the IOP sensing system are configured within a slit lamp to produce reliable results using equipment familiar to eye care professionals. In this way, IOP measurements using various embodiments of the disclosed IOP sensor and sensing system can take be easily obtained. Patients may also monitor their IOP in fine time resolution throughout their daily activities wherever a suitable reader is available.

Certain embodiments of the IOP sensor can include: a chamber having a first wall and a second wall opposing the first wall; a first array of photonic components inside of the chamber; at least one side wall separating the first and second walls; and an anti-reflective layer as part of or within the chamber.

In some embodiments, a raised portion is located within the chamber such that a height differential exists between an upper surface of the raised portion and the surface of the second wall. In some embodiments, the first array of photonic components is on a lower surface of the first wall.

Example embodiments of an apparatus for supporting an intraocular pressure sensor are also disclosed. The apparatus can include: a neck portion having an intraocular pressure sensor secured on a surface of the neck portion; a first arm coupled to a first side of the neck portion; and a second arm coupled to a second side of the neck portion, the first and second sides of the neck portion are opposed from each other.

Other systems, devices, methods, features and advantages of the subject matter described herein will be or will become apparent to one with skill in the art upon examination of the following figures and detailed description. It is intended that all such additional systems, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE DRAWINGS

The foregoing summary, as well as the following detailed description, is better understood when read in conjunction with the accompanying drawings. The accompanying drawings, which are incorporated herein and form part of the specification, illustrate a plurality of embodiments and, together with the description, further serve to explain the principles involved and to enable a person skilled in the relevant art(s) to make and use the disclosed technologies.

FIG. 1 is a perspective view of an example embodiment of an IOP sensor.

FIGS. 2-3 are cross-sectional views illustrating example embodiments of IOP sensors.

FIG. 4 is a process flow chart for an example method of signal-preprocessing, peak detection, reflectance spectra modeling and matching in accordance with example embodiments of the disclosure.

FIGS. 5A-5B are charts illustrating extrema matching and peak & valley detection processes in accordance with example embodiments of the disclosure.

FIGS. 6A-6B are charts illustrating air gap and pressure relationship according to an optomechanical model in accordance with example embodiments of the disclosure.

FIGS. 7A-B illustrate readout apparatuses with an integrated optical spectrometer and IOP measurement module in accordance with example embodiments of the disclosure.

FIGS. 8-10 illustrate exemplary IOP sensor and IOL combination apparatuses in accordance with example embodiments of the disclosure.

FIGS. 11A-E illustrate exemplary implantation procedures in accordance with example embodiments of the disclosure.

FIGS. 12-13 illustrate exemplary IOP sensors integrated with flexible support structures in accordance with example embodiments of the disclosure.

FIGS. 14 is a block diagram illustrating an example of a hardware implementation for an apparatus employing a processing system that may exploit the systems and methods of FIGS. 4-7 in accordance with some embodiments of the disclosure.

### DETAILED DESCRIPTION

### Overview

As previously mentioned, glaucoma is one of the most prevalent and perplexing disease today. By 2020, it is projected that 79.6 million people worldwide will have the disease. Of those 79.6 million, 4.5 million of them will suffer from irreversible bilateral vision loss. Currently, IOP is the most important modifiable risk factor for glaucoma. Moderating elevated IOP levels by individualized medication or surgery is the only available therapeutic modality. IOP typically has a circadian rhythm that fluctuates between 10-21 mmHg throughout the day. This makes it difficult to diagnose and suggest treatments to patients based on sparse IOP measurements taken at a clinic. For example, in one study, a peak IOP measurement from a continuous 24 hour-monitoring-period was on average 5mmHg higher than a peak IOP measurement taken at the clinic. This false low pressure reading at the clinic may result in improper treatment in 80% of the patients. As such there is a need for a fast and accurate IOP measurement sensor and method that could be implemented for convenient tracking of patient IOPs over periods of weeks, months, and years and can take place in the home, office, or other environments.

Current clinic-based IOP measurement technology such as Dynamic Contour Tonometry (DCT) is both expensive and bulky. DCT is capable of sampling 100 IOP values per second with ±1 mmHg sensitivity. However, it requires very high computer processing power, which is one of the reasons why DCT is expensive and bulky (large computer equipment). Additionally, although DCT systems can be sensitive, the IOP measurement is inaccurate because it is an indirect measure of pressure by tapping the cornea surface from the outside of the eye. Other existing IOP measurement technologies such as LC sensor implants, micro-fluidic channel sensors, and strain gauges lack the sensitivity and sampling time resolution compared to DCT. For example, LC sensor implants have a sensitivity of 2.5 mmHg and micro-fluidic channel sensors have a sensitivity of 0.5 mmHg but lacks the sampling speed and rate. Additionally, LC systems require large components (size of a standard microwave oven). Accordingly, there is a need for fast and accurate an IOP measurement technology that could be implemented in a more affordable fashion—without the need of expensive equipment.

Generally, IOP measurement technologies seek to achieve two performance goals: (1) high sensitivity: to measure IOP in sub-1 mmHg scale; and (2) high sampling frequency: to detect acute IOP fluctuations by obtaining an IOP profile with high temporal resolution. Provided herein are example embodiments of IOP systems and IOP measurement methods that are both highly sensitive and faster than conventional techniques. These embodiments can have a sensitivity of ±0.01 mmHg and microsecond level processing time per signal. This is a considerable improvement in both accuracy and speed over existing IOP measurement technologies (e.g., DCT, LC sensor, micro-fluidic channel, and strain gauges).

In certain embodiments disclosed herein, an intraocular pressure sensing system includes an implantable sensor for sensing intraocular pressure and a detection device (or a reader). The implantable IOP sensor may include a chamber with a first and second walls, and a plurality of photonic components adapted to reflect light. The first wall may be a flexible membrane made of a silicon-nitride. Within the chamber, a raised portion is provided on a surface of the second wall. The chamber may include an air gap formed by the spacing between the first membrane structure and the exposed surface of the raised portion. The flexible membrane is movable with respect to the raised portion in response to a change in ambient (surrounding) pressure. The IOP sensor may also include at least one sidewall separating the first and second walls. The at least one sidewall may be perpendicular to each of the first and second walls. Additionally, a layer of anti-reflection material may be provided between the at least one sidewall and the first wall. The anti-reflection coating may be black silicon or other suitable material that absorbs light at a broad range of wavelengths.

The IOP sensor has a resonance frequency that shifts as a size of the gap changes. The detection device can be adapted to transmit optical light to the IOP sensor and detect the resonance frequency of the IOP sensor based on at least one wavelength of light reflected from the IOP sensor. The detection device can be adapted to detect the resonance frequency based on a magnitude variation of the at least one wavelength of light reflected from the implantable device. The detection device can be adapted to determine the intraocular pressure based on the detected resonance frequency of the implantable IOP sensor. The detection device may be a standard slit lamp retrofitted with an optical spectrometer and an IOP sensing module. The detection device consisting of an optical spectrometer and IOP sensing module may also be located within household items such as mirrors, computer displays, TV screens. By leveraging standard/ubiquitous equipment, an accurate and inexpensive IOP sensing system is achieved.

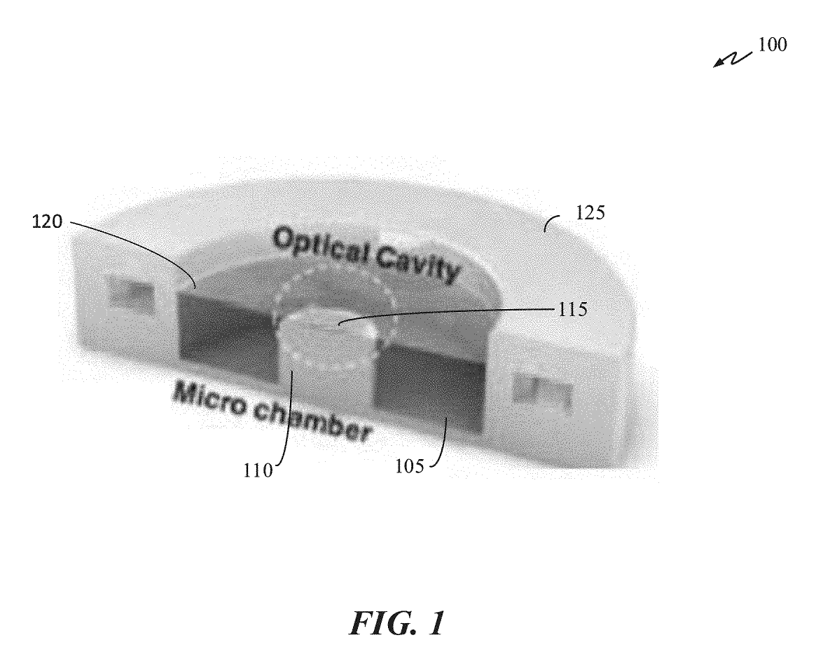

FIG. 1 illustrates an example embodiment of an IOP sensor 100 in accordance with aspects of the disclosure. IOP sensor 100 is a light-driven intraocular pressure sensing implant with a high sensitivity of ±0.01 mmHg. IOP sensor 100 can be placed between the cornea and the iris in the patient's eye as one example. To determine the intraocular pressure, IOP sensor 100 can be excited by an excitation beam (near-infrared light with wavelength between 700-1150 nm) from an external light source of a portable reader unit. The reflected light from the IOP sensor 100 can then be processed by an optical spectrometer located in the portal reader unit. The optical spectrometer is configured to determine a resonance spectrum from the reflected light.

The resonance spectrum can then be processed by an IOP measurement algorithm in four stages: (1) processing the resonance spectrum through a denoising and low pass filter, (2) detecting spectral feature (i.e., peaks and valleys), (3) fitting the best theoretical spectra for each experimental spectrum, and (4) calculating the intraocular pressure using an air gap and sensor pressure calculation based on a best-fit optomechanical model (OMM). The above algorithm is a vast improvement in accuracy and speed over conventional technologies. For example, a commercially available DCT system (with a conventional IOP measurement algorithm) is capable of sampling 100 IOP values per second, but requires high computer processing power. Additionally, DCT systems are bulky and inaccurate. The disclosed IOP measurement algorithm has a microsecond level processing time per signal (faster than current DCT system) without the need for an expensive and high-processing-power computer system.

### IOP Sensor

Referring again to FIG. 1, IOP sensor 100 can include a hermetically sealed cavity or chamber 105, a raised portion 110, an array of photonic components 115, a flexible membrane 120, and a protective section/layer 125. In some embodiments, chamber 105 may have a circular shape and may be further defined by a raised portion 110 at the center of chamber 105. Raised portion 110 may create a donut-shaped chamber. As shown in FIG. 1, raised portion 110 is shaped like a cylinder or has a circular cross-section. However, it should be noted that the cross-section of raised portion 110 may have other shape such as a square, a rectangle, an oval, etc. Chamber 105 may also exhibit other shape such as a square, a rectangle, a triangle, or an oval. Additionally, raised portion 110 may be centrally disposed at the center of chamber 105.

The array of photonic components 115 may be disposed on the bottom surface (facing the chamber) of the flexible membrane. Alternatively, the array of photonic components 115 may be disposed on a surface of raised portion 110. The photonic components described herein are components of the sensor and broadly serve to facilitate the reflection of light (photons) by the sensor. The photonic components, as will be described herein, can have various sizes and shapes, and can be arranged in arrays or patterns of various designs. In some embodiments, flexible membrane 120 may be made of silicon-nitride or other suitable material with low hysteresis. In this way, the deformation of the flexible membrane does not lag behind the intraocular pressure, which would give inaccurate pressure reading.

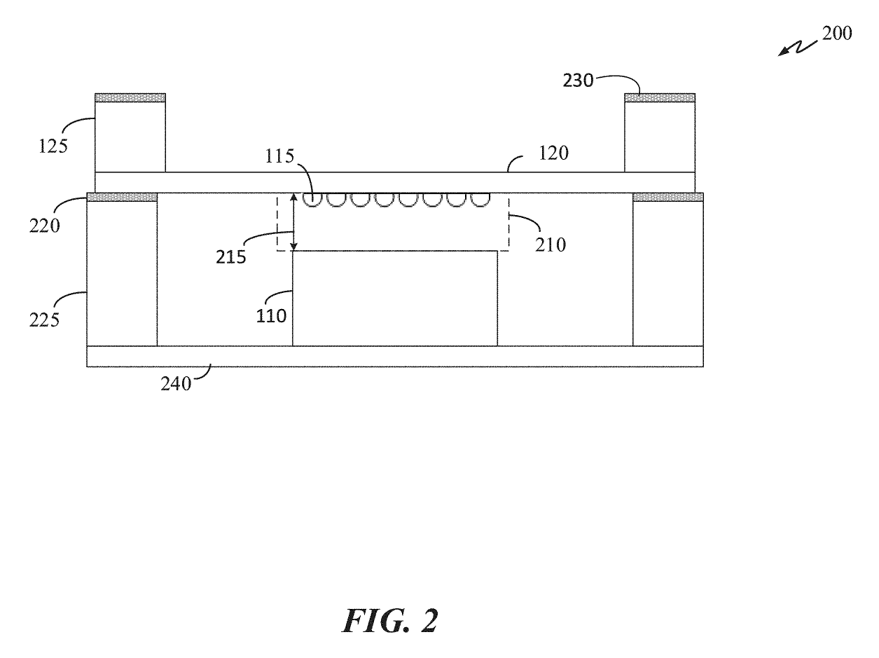

FIG. 2 illustrates a side view of IOP sensor 100 as shown in FIG. 1. IOP sensor 100 will now be described with reference to both FIGS. 1 and 2. In some embodiments, each photonic component of the array of photonic components 115 has a diameter between 100-1000 nm. In some embodiments, the diameter of each photonic component is 600 nm. Photonic array 115 may have an overall dimension of 200 μm by 200 μm, and the distance between the center of each photonic component may be between 500-1500 nm. In some embodiment, the distance between the center of each photonic component may be 1000 nm. The above noted diameters, overall dimensions, and the distances between the center of each photonic component are example dimensions of some embodiments and are not intended to be an exhaustive recitation of all dimensional variations present within the scope of this disclosure. To the contrary, other dimensions may be used while remaining within the scope of this disclosure.

Each photonic component may be globular, round, cylindrical, disk shaped, or curved in shape. In some embodiments, each photonic component has a hemispherical or substantially hemispherical shape, which has a wide angle of reflectance thereby enabling light to enter at a wide range angle of incidence. Those of skill in the field will readily recognize structures that are substantially hemispherical. In other words, the light source on the external reader does not have to be directly propagated (zero angle of incidence) at IOP sensor 100 (which can be implanted in the eye of a patient, or in a device that is implanted in the eye of a patient). One benefit to the hemispherical shape across the array is a smoother reflectivity distribution of the optical resonance spectrum as sharper transition edges (between each photonic component) lead to a deeper dip in the reflectivity distribution. This also contributes to a wider-band resonance spectrum and less sensitivity to the angle of incidence of the light source. In some cases, the photonic components can be referred to as nanodots, but this term does not require the components to be sized on a “nanoscale” nor have a round shape absent explicit recitation of such in the claims.

In some embodiments, cavity 105 may include an optical cavity 210, which can be formed by a distance 215 between the inner surface of flexible membrane 120 and the inner (and top) surface of raised portion 110. Distance 215 changes as flexible membrane 120 deforms toward or away from photonic array 115 due to the changes in the intraocular pressure. Distance 215 determines the distribution of the reflectance spectra. In turn, one or more variations (such as a peak or valley) of the resonance spectrum can be used to determine the intraocular pressure. In some embodiments, distance 215 has an initial distance range of 5-10 μm. In some embodiments, distance 215 may have an initial range of 7.3 μm. In some embodiments, the size of chamber 105 is the same as optical cavity 210. In these embodiments, raised portion 110 is not needed (does not exist) as the size of chamber 105 is the same size as optical cavity 210.

As shown in FIGS. 1 and 2, flexible membrane 120 forms one of the walls of chamber 105. In some embodiments, flexible membrane 120 may have a thickness between 0.2 to 0.5 μm. In another aspect, flexible membrane 120 has a thickness of 0.34 μm. At this thickness, the membrane's flexibility is optimum and the interference factor with infrared light is minimum. In some embodiments, flexible membrane 120 may be made of a silicon-nitrogen material such as SiN or Si₃N₄. Other transparent and flexible materials may also be used. However, at a minimum, the flexible material must not interfere with infrared light having wavelengths between 700 nm-1150 nm. The use of infrared light having wavelengths between 700 nm-1150 nm can be desirable because light at this range of wavelength is not detectable by the human eye as visible light may cause the patient to blink and/or squint. Additionally, near infrared light has excellent tissue penetration and its reflectance can be easily detected by a sensor.

Because flexible membrane 120 can be very thin and potentially fragile, protective section 125 may be disposed along the outer perimeter of membrane 115 but not to cover any portion of chamber 105. Protective layer 125 can be designed to give surgeons a surface to grab onto IOP sensor 110. This allows for the installation of IOP sensor 110 into a patient's eye without touching flexible membrane 120. In some embodiments, protective layer 125 may be made of silicon.

In some embodiments, an anti-reflection layer 220 may be included within or as part of chamber 105. In all embodiments, the anti-reflection layers can be applied as a coating (e.g., an anti-reflection coating) or can be introduced in a different fashion. Here, anti-reflection layer 220 is between a sidewall 225 and flexible membrane 120. In some embodiments, sidewall 225 is disposed along the perimeter of flexible membrane 120. Sidewall 225 may be one continuous wall. Alternatively, sidewall 225 may two or more walls coupled together. Additionally, sidewall 225 may be perpendicular to flexible membrane 120 and to a wall 240. In some embodiments, another layer of anti-reflection coating 230 is disposed on a surface (parallel to flexible membrane 120) of protective layer 125.

Anti-reflection coatings 220 and 230 may be made of silicon grass (black silicon). Alternatively, anti-reflection coatings 220 and 230 may be made of any suitable material that absorbs light at wavelength between 700 nm-1150 nm. This is the same wavelength range of the light source. In this way, only light from photonic array 115 is reflected back to the spectrometer, and any scattered light is absorbed by the anti-reflection coating.

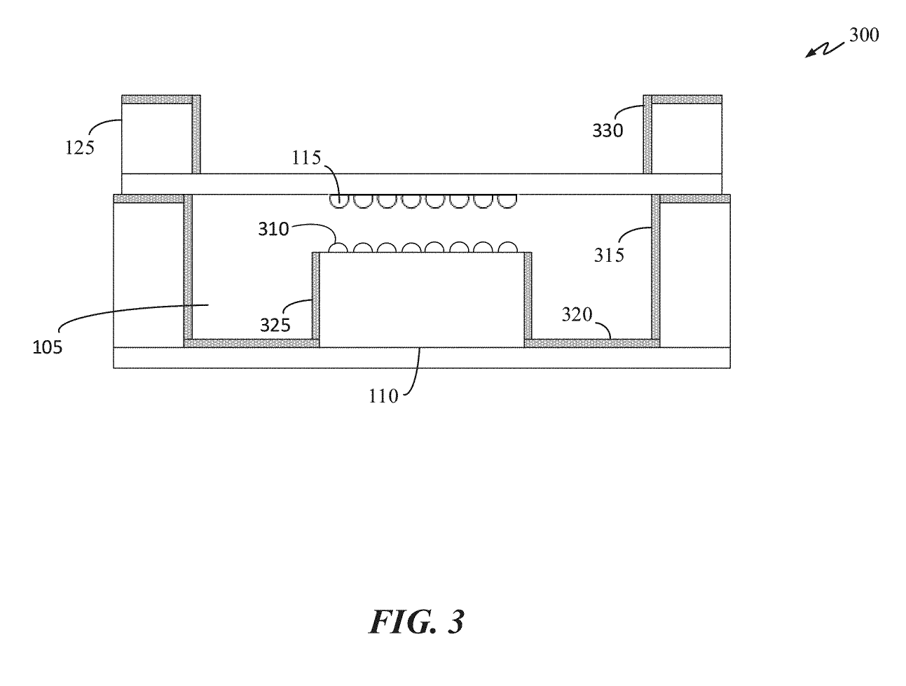

FIG. 3 illustrates an IOP sensor 300 in accordance with certain embodiments of the disclosure. IOP sensor 300 may include one or more features of IOP sensor 200 as previously described but with the addition of a second array of photonic components 310 disposed on the surface of raised portion 110. In some embodiments, photonic array 310 may be hemispherical or fully spherical. Additionally, photonic array 310 may have similar features exhibited by photonic array 115 as described above.

In some embodiments, IOP sensor 300 may include an anti-reflection coating on one or more of the interior walls of chamber 105. For example, chamber 105 may have one or more of anti-reflection coatings 315, 320, and 325 as shown in FIG. 3. Additionally, raised section 125 may include another anti-reflection coating 330 on its sidewall. In this way, the signal-to-noise ratio of IOP sensor 300 may be further increased due to the absorption of errant and/or scattered lights.

### IOP Measurement Algorithm

To obtain IOP measurements, an illuminant probe can be configured to shine near-infrared (NIR) light at IOP sensor 100. The NIR light may have a wavelength between 700-1150 nm. In this range, the NIR light is invisible to the human eye. Additionally, this range of wavelength has an excellent tissue penetration property. Because IOP sensor 100 may incorporate hemispherical photonic component (nanodot) array, the illuminant probe does not have to shine the NIR light directly perpendicular at sensor 100. The hemispherical shaped nanodot array enables the array to receive light at a wider range of angle of incidence and still can reflect light back at a near perpendicular angle with respect to the horizontal plane of the nanodot array.

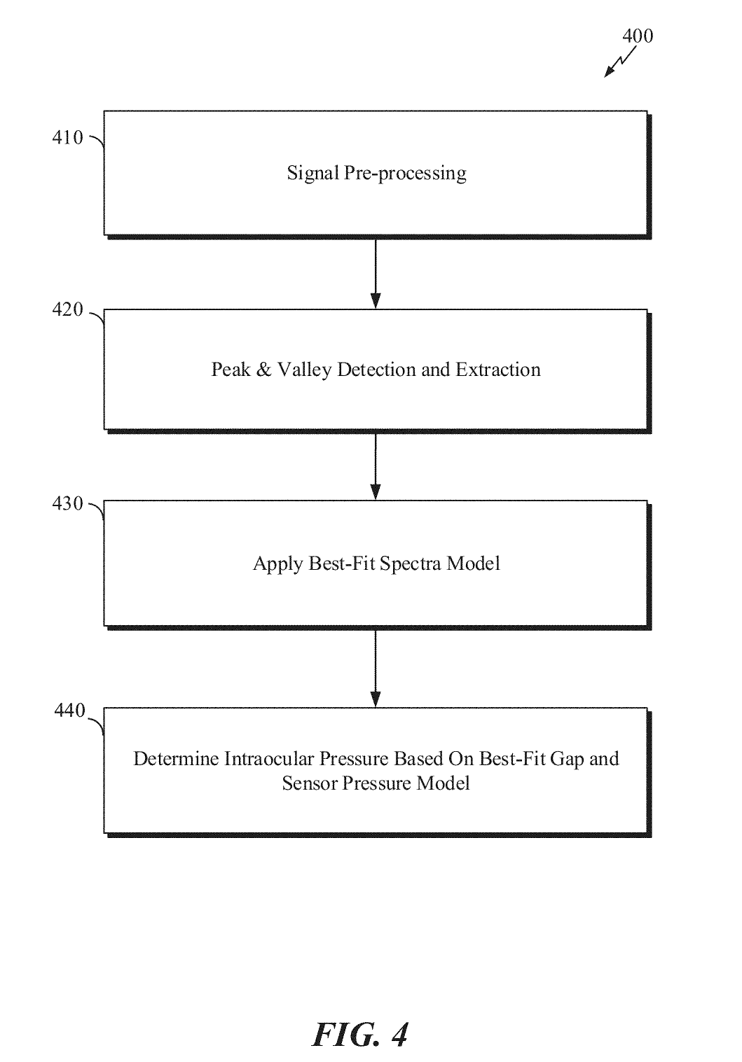

In operation, the reflected light (also referred to as a reflection spectra) is then captured by an optical spectrometer, which feeds its output to an IOP measurement module. FIG. 4 illustrates an example embodiment of an IOP measurement process 400 of IOP measurement module in accordance with some embodiments of the disclosure. Each and every step described with respect to FIG. 4 can be carried out by the use of one or more instructions stored in non-transitory memory of the system, for example within the reader or another computing device, that when executed by processing circuitry of the system, causes the step to be performed. The processing circuitry can be within one device, such as a microprocessor or ASIC, or can be distributed across multiple devices with processing capability in the system. Likewise, the instructions can be stored in one non-transitory memory device or can be separated in multiple non-transitory memory devices that each can be a discrete memory device or part of another device (e.g., a microprocessor or ASIC).

At stage 410, denoising and low pass filtering can be performed on the input signal—the reflection spectra. This preprocessing step can be performed to decrease noise and to identify and compensate for misalignments between the illuminant probe and IOP sensor 100. The first step of stage 410 may include a misalignment categorization process to categorize the misalignment between the illuminant probe and the IOP sensor using an alignment module. By appropriately categorizing the misalignment, the alignment module can instruct the user to make the appropriate correction in real-time in order to achieve a better or optimum signal. Generally, there are two orientation conditions—transverse and longitudinal—that should be met in order to obtain proper resonance spectra. First, the light probe should be fixated at a transverse coordinate within a valid focal range in order to prevent over and under-reflection. Second, the convergence point of the light should be in close proximity to the sensor diaphragm center in order to mitigate peripheral reflections from the cavity contour. When these three positioning conditions are not satisfied, the reflection spectrum becomes saturated, which will overwhelm optical sensors in the spectrometer. Alternatively, incorrect probe positioning may also lead to no signal or cause the reflection spectra to have a single peak waveform resulting from the black-body radiation profile of the light source.

In some embodiments, when a saturated signal is detected, the alignment module may generate an instruction to instruct the user to adjust the focal length of the illumination light source to be shorter. In other words, a saturated signal indicates that the focal point the illumination light source is too long and extends beyond optical cavity of IOP sensor 100. Alternatively, when no signal is detected, the alignment module may generate an instruction to direct the user to adjust the focus length and make it shorter. A no signal indicates that the focal length of the light source is too short. Additionally, when a single peak spectra occurs, an instruction can be generated to direct the user to translate the probe in the horizontal direction with respect to the eye or IOP sensor 100. In some embodiments, the instruction generated to the user may be audio, textual, graphical, or a combination thereof.

In some embodiments, the alignment module can be modified to recognize misalignment waveforms such as saturation, single peak intensity, or no signal. For example, minima detection may be converted into a peak detection problem by inverting the spectrum. In some embodiments, a minimum peak prominence threshold may be set to detect both the peak intensity and low prominence peak (no signal), both of which indicate a misalignment. Further, the gradient of the intensity spectrum may be swept to check for extended regions having zero slope to filter out misalignment caused by saturation. In some embodiments, the peak detection function (of the alignment module) may output the location of the valleys that met the threshold requirements. If no valleys were detected, the optical detector and IOP sensor are misaligned. In this case, the main program terminates with an IOP value of zero and the alignment module informs the user of a misalignment in the set up. If more than one valley was detected, the algorithm proceeds to input the extracted valleys.

In some embodiments, the illumination probe may be coupled to one or more servomotors or linear actuators and IOP process 400 may be configured to send instruction to a controller to automatically adjust the illumination probe based on the alignment analysis. In some embodiments, the illumination probe and the one or more servomotors and/or linear actuators may be coupled to a standard slit lamp.

As mentioned, part of the alignment detection algorithm includes spectral feature recognition algorithm that detects peaks and valleys. At stage 420, on a high level, if the valleys are found to meet the detection thresholds, the alignment algorithm may conclude that the setup is well-aligned and proceed to the next step.

In some embodiments, three thresholds were integrated into the peak detection in order to track the desired valley locations. A minimum peak prominence (p_min) threshold was set in order to filter out misalignment (ii) and (iii) both of which display a single low prominence peak. Second a minimum peak distance (d_min) threshold was applied to deter remnant noise in the vicinity of one peak from being detected as multiple peaks. Lastly, the gradient of the intensity spectrum was swept to check for extended regions having zero gradients to filter out misalignment (i).

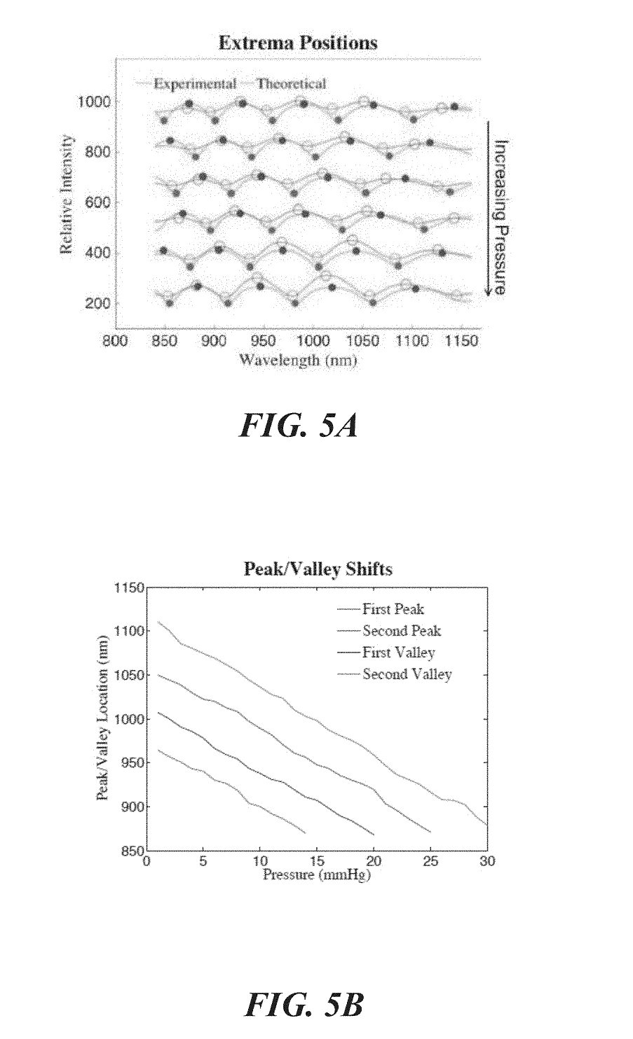

In some embodiments, the signal demodulation algorithm at stage 420 includes a peak detection function configured to output the location and prominence of the valleys found satisfying the requirements set by the thresholds, which can be the minimum peak prominence threshold. FIG. 5A illustrates how experimental results match well with theoretical model of the alignment detection module. As shown in FIG. 5, extrema positions of theoretical model closely track to extreme positions from experimental results. This allows process 400 to accurately map out the relationship between pressure and peak & valley locations of the resonance spectra.

FIG. 5B illustrates the peak/valley and pressure relationship derived from the optomechanical model of process 400. As shown in FIG. 5B, that there is a highly linear behavior between the locations of the extrema and the increasing pressure. A nearly linear shift in wavelength of prominent features may be observed by tracking each set of peaks/valleys with respect to increasing pressure.

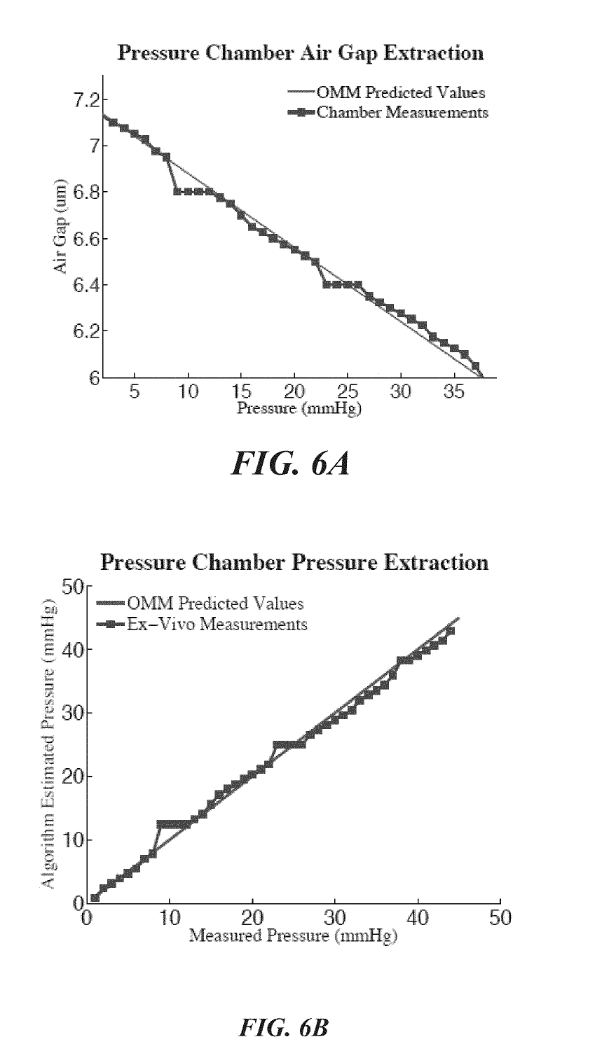

To map the IOP levels from the reflection spectra, process 400 applies the Extrema-Matching technique combined with optomechanical modeling of FIG. 5B. In the optomechanical model, a mechanics model predicts the deformation of the sensor membrane with increasing pressure while the resonance model generates the reflection spectrum as a function of deformation. Using the optomechanical model, process 400 can predict the corresponding air gap for each measured spectrum as shown in FIG. 6A. A correlation between the experimental and simulated sensor air gap was found to be −0.9973 (p<0.01). Finally, based on the mechanics model, the air gap is mapped to the pressure as shown in FIG. 6B. This approach correctly identifies IOP in 95.5% of measurements within a ±2 mmHg error.

It should be noted that the algorithms or instructions of IOP measurement process 400, which includes a signal demodulation, valley/peak detection, and spectral features recognition and matching may be stored on a memory that is readable by a computer. A computer may be a processor or an application specific integrated circuit (ASIC). When the algorithms or instructions are executed by the computer, the instructions will cause the computer to carry out the functionalities of IOP measurement process 400 as described above.

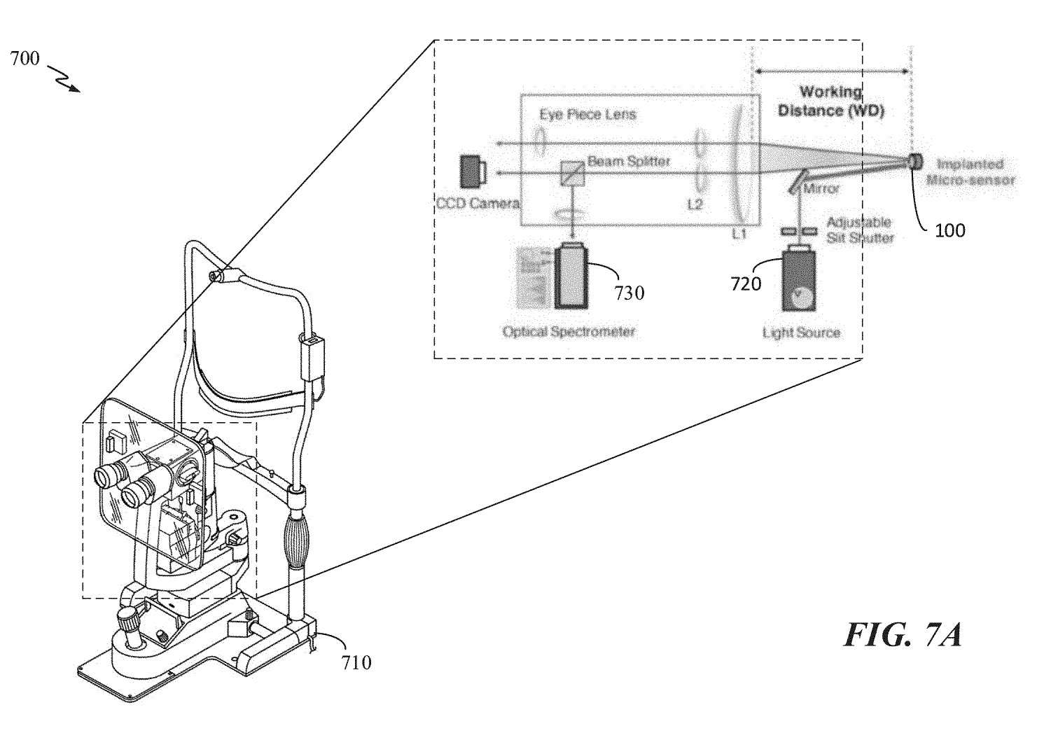

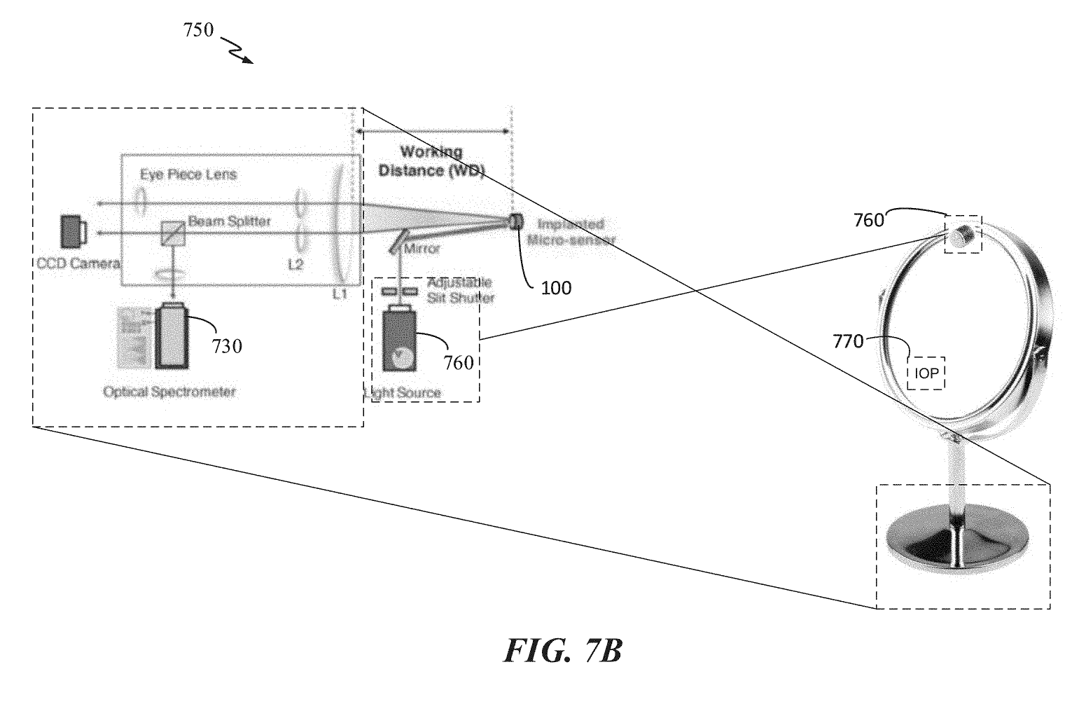

FIG. 7A illustrates an optical readout apparatus 700 in accordance with an aspect of the disclosure. Apparatus 700 includes a standard slit lamp 710 having an integrated illumination probe 720 and an optical spectrometer 730. In some embodiments, illumination probe 720 may be a commercial 6-around-1 specular reflectance probe having fiber bundle in conjunction with a collimating lens to provide both illumination and detection capabilities.

In some embodiments, a broadband light source in the visible or the invisible spectrum can be used. Illumination probe 720 can be configured to illuminate the implanted sensor using broadband light and then detect the reflection from the sensor. For IOP readout, the reflected light may be relayed to optical spectrometer 730 embedded within apparatus 700. In some embodiments, apparatus 700 also includes a display (not shown) for displaying the IOP readout and/or the resonance spectra of the reflectance light. In some embodiments, optical spectrometer 730 is not integrated with slit lamp 710, but instead is optically coupled to slip lamp 710 using, for example, optical fibers.

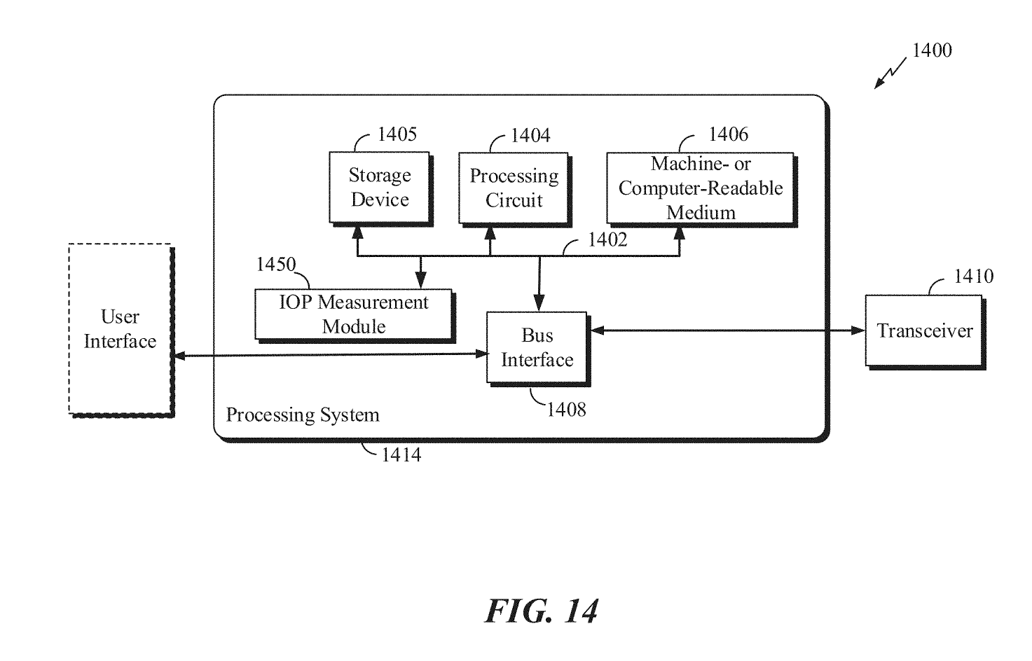

In some embodiments, apparatus 700 includes an ASIC (see FIG. 14) that is configured to execute IOP process 400, which includes algorithms for denoising, peak & valley detection module, alignment module, reflectance spectra modeling & matching module, and computing the IOP based on the resonance spectra peak/valley profile. Alternatively, apparatus 700 may include a communication module (not shown) configured to send the output (i.e., resonance spectrum) of the optical spectrometer to a remote server for IOP analysis. Once the analysis is completed, the results may be received by the communication module of apparatus 700 for display. In this way, apparatus 700 may be inexpensively produced for home use.

FIG. 7B illustrates an optical readout apparatus 750 in accordance with an aspect of the disclosure. As shown in FIG. 7B, readout apparatus 750 may be a desk mirror. Alternatively, readout apparatus 750 may be any home device such as a wall mirror, a television, a handheld mirror, a tablet, a pair of glasses, a virtual reality headset, a helmet, or any other device capable of integrating an optical spectrometer and a display. Readout apparatus 750 may include an integrated light source (e.g. illumination probe) configured to shine light directly into the user's eye and into the optical cavity of an IOP sensor (e.g., IOP sensor 100, 200, and 300). In some embodiments, optical spectrometer 730 may be integrated into the base of desk mirror 750. Similar to apparatus 700, readout apparatus 750 may include an ASIC configured to execute IOP process 400, which includes algorithms for denoising, peak & valley detection module, alignment module, reflectance spectra modeling & matching module, and computing the IOP based on the resonance spectra peak/valley profile. In some embodiment, apparatus 750 include a communication module or an ASIC (e.g., a transceiver) configured to send the output of optical spectrometer 730 to a remote server for IOP analysis. The remote server may include a processor and algorithms (instructions), when executed, cause the processor to execute IOP process 400 which includes various processes as described above. Once the remote server completes the IOP analysis, the results are sent back to the communication module of apparatus 750, which may display the results on a display area 770 of apparatus 750.

### IOP Sensor on Intraocular Lenses

Many patients with glaucoma develop lens cataract and have to undergo lens removal followed by intraocular lens (IOL) implantation in order to improve their vision. During a cataract surgery, IOL is implanted into the empty capsular bag to restore some of the function of the native lens. Although the IOL technology is well developed, there is no option in the market place for an IOL with an integrated IOP sensor. An IOL is typically well-centered and mechanically stable within the capsular bag once implanted. The IOP sensor can be piggy-backed onto an IOL and implanted into patients with lens cataract as well as glaucoma. Further, an IOP sensor-IOL combo apparatus would greatly decrease the chances of implantation error and damage to the IOP sensor during implantation by having the IOP sensor pre-integrated with an IOL. This also ensure stable placement of the IOP sensor on the IOL and thereby reducing the chances of obscuring the patient's vision caused by erroneous placement.

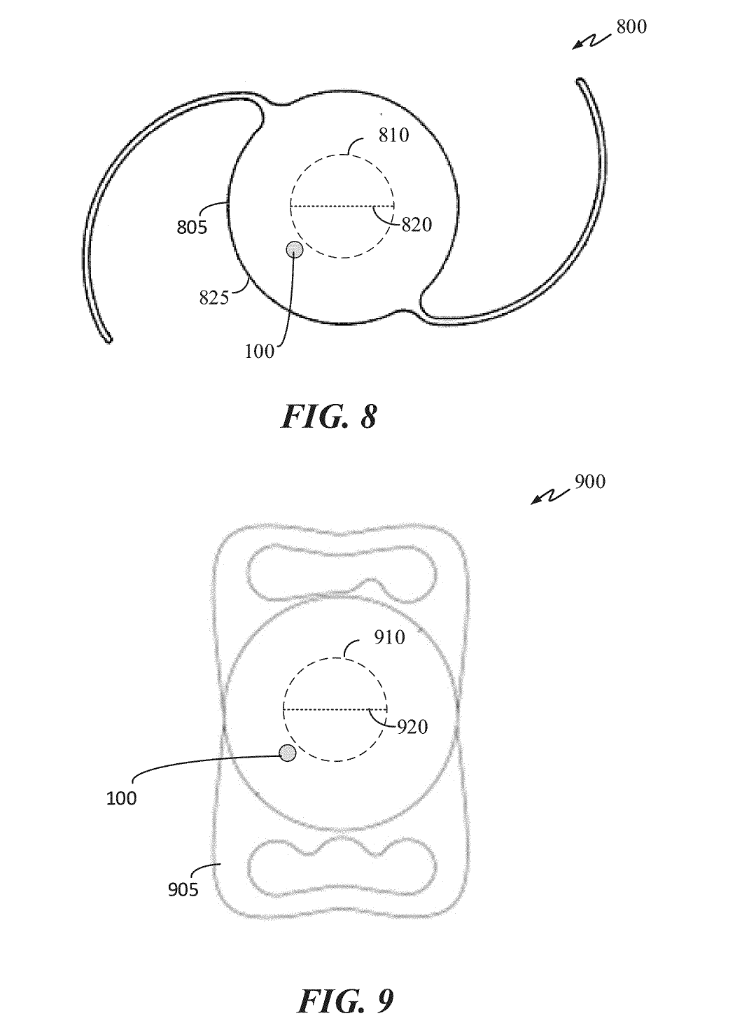

FIG. 8 illustrates an IOP sensor-lens combo apparatus 800 in accordance with some embodiments of the disclosure. Apparatus 800 includes an intraocular lens 805 and an IOP sensor 100 (or IOP sensor 200) that is placed within a certain distance from the center of lens 805. As shown in FIG. 8, lens 805 has a center region 810 having a diameter 820 of approximately 3 mm. In some embodiments, IOP sensor 100 is disposed just outside of region 810. In this way, the vision of the patient is not obscured by IOP sensor 100. In some embodiments, IOP sensor 100 is placed at a distance between 1.25 to 2 mm from the center of lens 805. It should be noted that it is not desirable to place IOP sensor too close to an outer edge 825 of lens 805 as the thickness of the lens may be insufficient to properly support IOP sensor 100. At the same time, IOP sensor should not be placed too close to the center of lens 805 as it would obscure the patient's vision. In some embodiments, lens 805 has a thickness range between 250-350 microns.

IOP sensor 100 may be adhesively attached to lens 805 using a medical grade silicone adhesive. Alternatively, lens 805 may be configured to have a pocket or slot (not shown) that would provide a tight fit for IOP sensor 100. Other alternatives include physical features on the sensor and on the IOL that will mechanically fit together, locking in the sensor onto the IOL. In some embodiments, IOP sensor 100 may be adhesively attached to the bottom of a slot of lens 805. The slot may be configured to provide a tight fit but not to completely encase the IOP sensor. In some embodiment, the slot would leave flexible membrane 120 of IOP sensor 100 facing outwards of the eye. In this way, IOP sensor 100 can properly receive light from an illumination probe and to measure the intraocular pressure. Similarly, when adhesive is used to affix IOP sensor 100 onto lens 805, the adhesive is applied on the bottom surface of IOP sensor 100, which is the surface on the opposite side of flexible membrane 120. The side where flexible membrane 120 and photonic array 115 can be seen is referred to the front surface. Accordingly, when affixing IOP sensor 100 to lens 805, only the bottom surface is in contact with the surface of lens 805—leaving the front surface an obstructed.

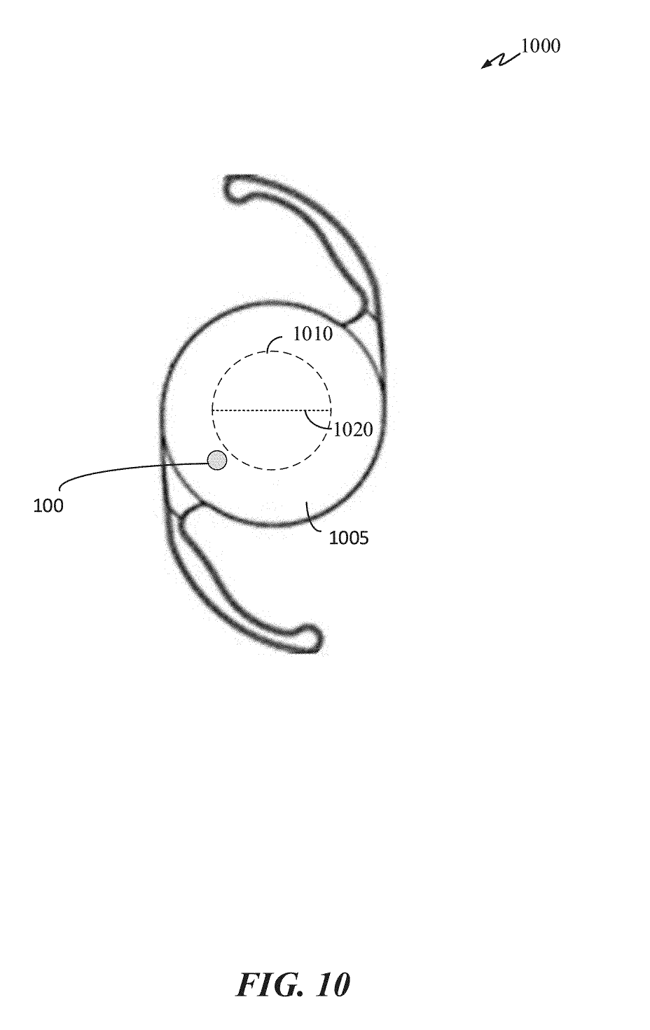

FIGS. 9 and 10 illustrate IOP sensor-lens combo apparatuses 900 and 1000 in accordance with some embodiments of the disclosure. Apparatus 900 includes a lens 905 and IOP sensor 100. As in the case of apparatus 800, IOP sensor 100 should be placed just outside of a center region 910 of lens 905. In some embodiments, center region 910 has a diameter range between 2 mm and 4 mm. Similarly, apparatus 1000 includes lens 1005 and IOP sensor 100 being disposed just outside of a center region 1010, which as a diameter of approximately 3 mm. Although apparatuses 800, 900 and 1000 are described to contain IOP sensor 100, each may contain IOP sensor 200 in place of IOP sensor 100.

In some embodiments, IOP sensor may be integrated with other ophthalmic devices such as minimally invasive glaucoma surgery (MIGS) device. MIGS devices are tube-like units that drain intraocular aqueous fluid into a variety of anatomical compartments in the eye to relieve high intraocular pressure. To facilitate this mode of attachment, the IOP sensor may have curved legs attached to its bottom surface of the sensor 100. Such legs can be used to mechanically fit around the tubular structure of the MIGS device.

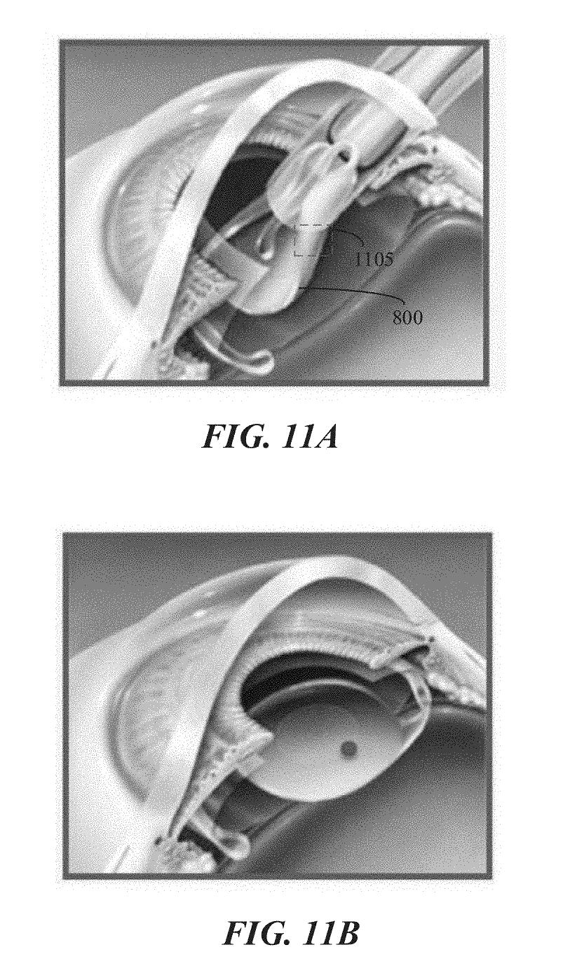

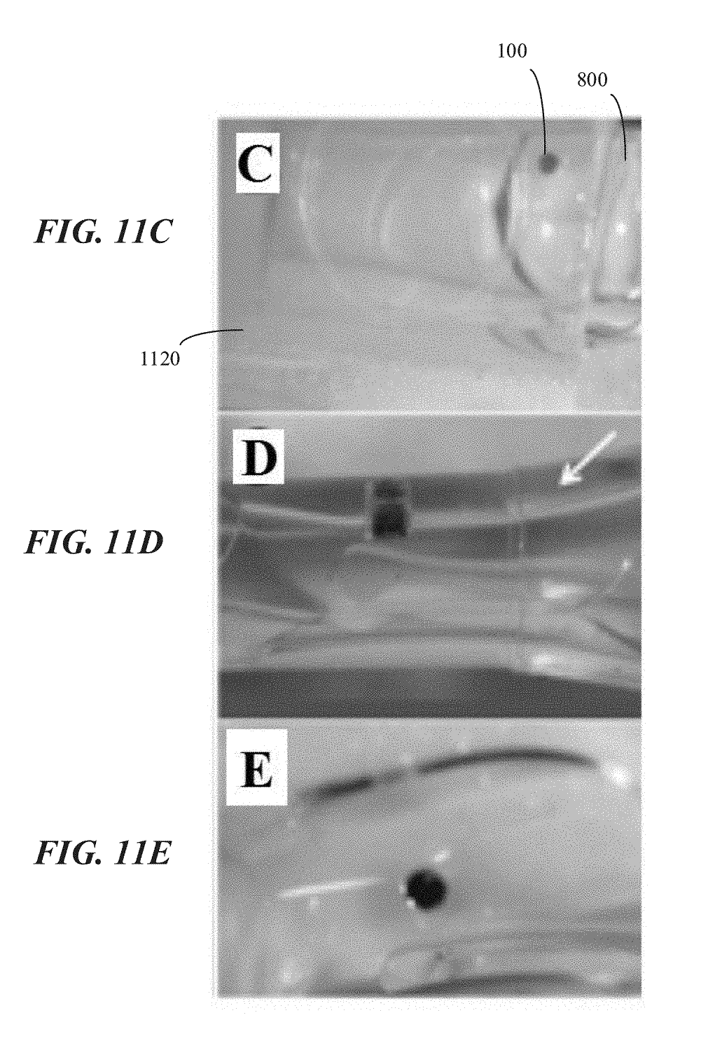

FIGS. 11A-E illustrate the implantation procedure of apparatus 800, 900, or 1000. FIG. 11A shows an IOP Sensor-IOL combo apparatus (e.g., apparatus 800, 900, or 1000) rolled up into a taco-shaped position. Although not shown in FIG. 11A, IOP sensor 100 is located on the inner side of the taco-shaped fold in an area 1105 where the surface is flat or the curvature of the fold is at a minimum (see FIG. 11B). Once the IOL is placed inside of the eye (e.g., into the capsular bag, sulcus, or affixed to the iris etc.), apparatus 800 is unfolded into position (FIG. 11B) with the front side of IOP sensor facing toward the exterior of the eye.

FIGS. 11C-E illustrate a rolling up procedure of IOP sensor-IOL combo apparatus using an inserter capable of implanting an IOP sensor-IOL combo apparatus through a very small incision. FIG. 11C shows an inserter tube 1120 with apparatus 800 folded into a cylindrical position with IOP sensor 100 on the inside of the folded IOL. In this way, IOP sensor 100 is protected by the IOL of apparatus 800. It should be noted that the front side of IOP sensor 100 is facing toward the inner portion of the cylinder (toward the main axis of the cylindrical fold). In this way, IOP sensor 100 is protected during the implantation procedure. As previously noted, the front side of IOP sensor 100 is the side where flexible membrane 120 and photonic array 115 are facing the outside of the eye (can be visually inspected from outside of the eye).

FIG. 11D shows apparatus 800 being further advanced into the inserter tube. FIG. 11E shows apparatus 800 beginning to unfold as it passes out of the inserter tube, for example into the capsular bag within the eye. Once out of the confined space within the inserter tube, the IOL with IOP sensor unfolds itself, resulting in the IOP sensors flexible membrane 120 facing outwards of the eye.

### IOP Sensor on Flexible Support

Sometime a patient with glaucoma or a risk of having glaucoma only needs an IOP sensor and cataract surgery with IOL implantation is not needed. In this case, one innovative way to efficiently and accurately implant an IOP sensor is to provide the IOP sensor with a set of flexible supports for fixation within the anterior chamber. IOP sensors with such flexible supports may be implanted into the anterior chamber, allowing the supports to hold the senor in place. The flexible support provides several advantages over implanting only an IOP sensor. First, the flexible support provides a surface for securing the IOP sensor into a desirable position since the IOP sensor is affixed at a predetermined position on the flexible support. This takes the guess work away from surgeons as there is no requirement to precisely place the IOP sensor within the eye. Secondly, the flexible support provides a surface for the surgeon to grab and manipulate the IOP sensor-flexible support combo apparatus into position without having to physically touch the IOP sensor. Additionally, the flexible support, when folded during the implantation procedure, provides a protective structure that surrounds the IOP sensor. Further, other advantages include faster implantation procedure with fast tissue recovery; and easy and fast retrieval of the IOP sensor if needed.

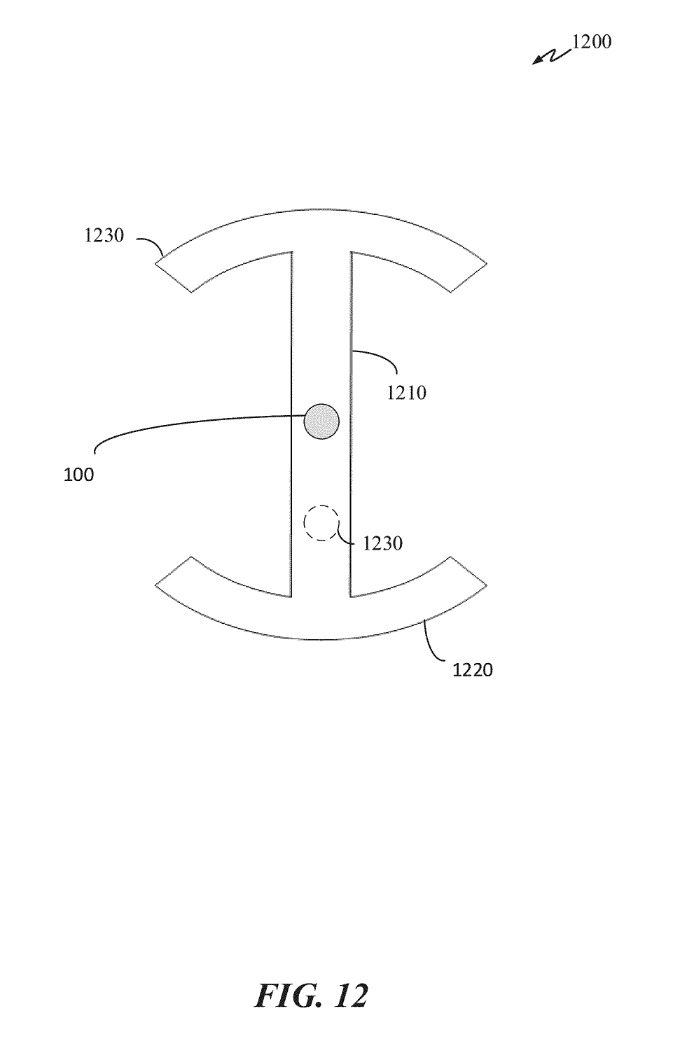

FIG. 12 illustrates an IOP sensor/flexible support combo apparatus 1200 in accordance with some embodiments of the disclosure. Apparatus 1200 includes an IOP sensor (e.g., IOP sensor 100 or 200), a neck portion 1210, a first arm portion 1220, and a second arm portion 1230. IOP sensor 100 may be attached to neck portion 1210 using adhesive or mechanical means such as a pocket or slot (friction hold) in neck portion 1210. In some embodiments, first arm portion 1220 is located at one end of neck portion 1210 and second arm portion 1230 is located at the opposite end of neck portion 1210. Each of the arm portion may have an arc-shaped structure with a concave toward neck portion 1210. Each arm may be configured to have sufficient mechanical strength to lay relatively flat so to not hug the curvature of the eye and potentially irritate the endothelium layer of the cornea. In this way, contact or interference with the eye tissues is minimized. In some embodiments, each arm may have a straight shape rather than an arc shape. Apparatus 1200 may be formed from thin medical grade silicone sheet material and may have a thickness range of 50-400 microns or any other suitable flexible and biocompatible material. Apparatus 1200 may have 2 or more legs and their arc shaped structures that will be in close approximation with the eye's angle tissue may be of other shapes to minimize contact with angle tissue.

In some embodiment, neck portion 1210 may be curved in order to avoid obscuring the center region of the eye. The curve area of neck portion 1210 may have a diameter of at least 3 mm. In some embodiments, IOP sensor 100 may be located near the center of neck portion 1210. Alternatively, IOP sensor 100 is off centered near one of the arms at location 1230.

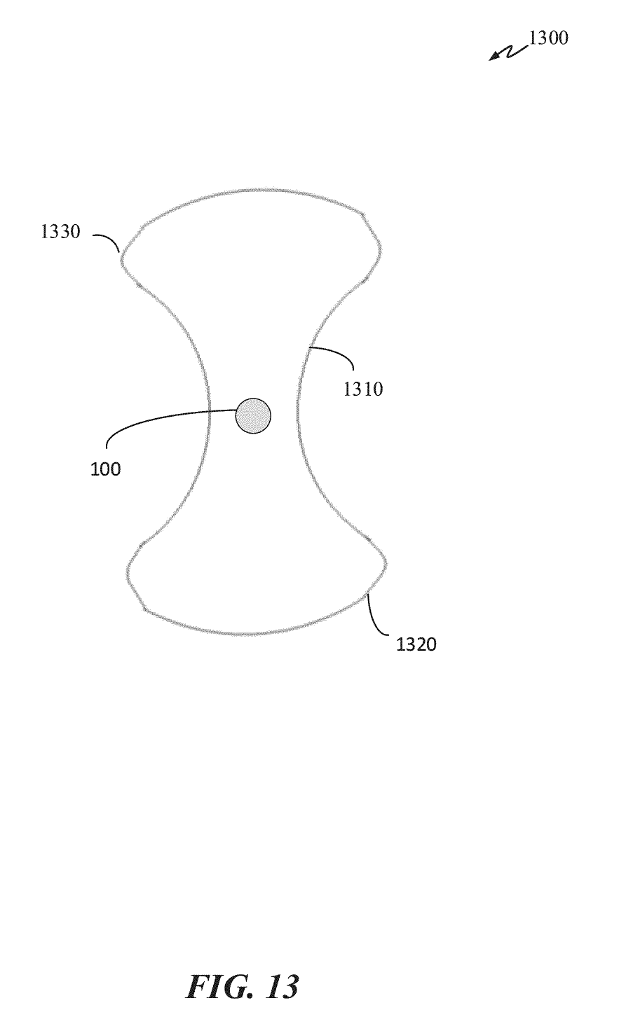

FIG. 13 illustrates an IOP sensor/flexible support combo apparatus 1300 in accordance with some embodiments of the disclosure. Apparatus 1300 includes a neck portion 1310, a first arm portion 1320, and a second arm portion 1330. As shown in FIG. 13, apparatus 1300 may have a bow shaped (or hour-glass) structure with each arm shapes like a wedge with rounded outer edges. Neck portion 1310 may include an IOP sensor (e.g., sensor 100 or 200) at the center. Alternatively, sensor 100 may off center at location 1330. The IOP sensor may be affixed using adhesive or mechanical means such as a pocket or slot. Similar to apparatus 1200, apparatus 1300 may be made of medical grade silicone or other flexible material suitable to be implanted in the eye. Apparatus 1300 may have one or more features of apparatus 1200 as described with respect to FIG. 12.

### Example Software Module/Engine and Hardware Implementation

FIG. 14 illustrates an overall system or apparatus 1400 configured to execute IOP measurement process 400. In accordance with various aspects of the disclosure, an element, or any portion of an element, or any combination of elements may be implemented with a processing system 1414 that includes one or more processing circuits 1404. Processing circuits 1404 may include micro-processing circuits, microcontrollers, digital signal processing circuits (DSPs), field programmable gate arrays (FPGAs), programmable logic devices (PLDs), state machines, gated logic, discrete hardware circuits, and other suitable hardware configured to perform the various functionality described throughout this disclosure. That is, the processing circuit 1404 may be used to implement process 400 described above and illustrated in FIGS. 4 through 16.

In the example of FIG. 14, the processing system 1414 may be implemented with a bus architecture, represented generally by the bus 1402. The bus 1402 may include any number of interconnecting buses and bridges depending on the specific application of the processing system 1414 and the overall design constraints. The bus 1402 links various circuits including one or more processing circuits (represented generally by the processing circuit 1404), the storage device 1405, and a machine-readable, processor-readable, processing circuit-readable or computer-readable media (represented generally by a non-transitory machine-readable medium 1406.) The bus 1402 may also link various other circuits such as timing sources, peripherals, voltage regulators, and power management circuits, which are well known in the art, and therefore, will not be described any further. The bus interface 1408 provides an interface between bus 1402 and a transceiver 1410. The transceiver 1410 provides a means for communicating with various other apparatus over a transmission medium.

The processing circuit 1404 can be responsible for managing the bus 1402 and for general processing, including the execution of software module/engine stored on the machine-readable medium 1406. In some embodiments, IOP measurement module 1450 include algorithms as described in process 400, when executed by processing circuit 1404, causes processing system 1414 to perform the various functions described herein for any particular apparatus. Machine-readable medium 1406 may also be used for storing data that is manipulated by processing circuit 1404 when executing software module/engine.

One or more processing circuits 1404 in the processing system may execute software module/engine or software module/engine components. Software module/engine shall be construed broadly to mean instructions, instruction sets, code, code segments, program code, programs, subprograms, software module/engines/engines, applications, software module/engine applications, software module/engine packages, routines, subroutines, objects, executables, threads of execution, procedures, functions, etc., whether referred to as software module/engine, firmware, middleware, microcode, hardware description language, or otherwise. One or more processing circuits (or processing circuitry) may perform the tasks. A code segment may represent a procedure, a function, a subprogram, a program, a routine, a subroutine, an engine, a module, a software module/engine package, a class, or any combination of instructions, data structures, or program statements. A code segment may be coupled to another code segment or a hardware circuit by passing and/or receiving information, data, arguments, parameters, or memory or storage content. Information, arguments, parameters, data, etc. may be passed, forwarded, or transmitted via any suitable means including memory sharing, message passing, token passing, network transmission, etc.

The software module/engine may reside on machine-readable medium 1406. The machine-readable medium 1406 may be a non-transitory machine-readable medium. A non-transitory processing circuit-readable, machine-readable or computer-readable medium includes, by way of example, a magnetic storage device (e.g., hard disk, solid-state drive), an optical disk (e.g., a compact disc (CD) or a digital versatile disc (DVD)), a smart card, a flash memory device (e.g., a card, a stick, or a key drive), RAM, ROM, a programmable ROM (PROM), an erasable PROM (EPROM), an electrically erasable PROM (EEPROM), a register, a removable disk, a hard disk, a CD-ROM and any other suitable medium for storing software module/engine and/or instructions that may be accessed and read by a machine or computer. The terms “machine-readable medium”, “computer-readable medium”, “processing circuit-readable medium” and/or “processor-readable medium” may include, but are not limited to, non-transitory media such as portable or fixed storage devices, optical storage devices, and various other media capable of storing, containing or carrying instruction(s) and/or data. Thus, the various methods described herein may be fully or partially implemented by instructions and/or data that may be stored in a “machine-readable medium,” “computer-readable medium,” “processing circuit-readable medium” and/or “processor-readable medium” and executed by one or more processing circuits, machines and/or devices. The machine-readable medium may also include, by way of example, a carrier wave, a transmission line, and any other suitable medium for transmitting software module/engine and/or instructions that may be accessed and read by a computer.

The machine-readable medium 1406 may reside in the processing system 1414, external to the processing system 1414, or distributed across multiple entities including the processing system 1414. The machine-readable medium 1406 may be embodied in a computer program product. By way of example, a computer program product may include a machine-readable medium in packaging materials. Those skilled in the art will recognize how best to implement the described functionality presented throughout this disclosure depending on the particular application and the overall design constraints imposed on the overall system.

One or more of the components, steps, features, and/or functions illustrated in the figures may be rearranged and/or combined into a single component, block, feature or function or embodied in several components, steps, or functions. Additional elements, components, steps, and/or functions may also be added without departing from the disclosure. The apparatus, devices, and/or components illustrated in the Figures may be configured to perform one or more of the methods, features, or steps described in the Figures. The algorithms described herein may also be efficiently implemented in software module/engine and/or embedded in hardware.

Note that the aspects of the present disclosure may be described herein as a process that is depicted as a flowchart, a flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed. A process may correspond to a method, a function, a procedure, a subroutine, a subprogram, etc. When a process corresponds to a function, its termination corresponds to a return of the function to the calling function or the main function.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1. An intraocular pressure sensing system comprising:
a chamber having a first wall and a second wall opposing the first wall, wherein the chamber comprises an anti-reflection layer;
a first array of photonic components disposed inside of the chamber; and
at least one sidewall separating the first and second walls.

2. The intraocular pressure sensing system of claim 1, further comprising:
apparatus having a light source configured to shine light into the chamber;
an optical spectrometer configured to receive light reflected from the first array of photonic components inside of the chamber and to output a signal indicative of a resonance spectrum upon receiving the reflected light;
a processor coupled to the optical spectrometer; and
non-transitory memory on which is stored a plurality of instructions that, when executed, cause the processor to determine the intraocular pressure based on the signal indicative of a resonance spectrum.

3. The intraocular pressure sensing system of claim 1, wherein the first wall comprises a flexible membrane.

4. The intraocular pressure sensing system of claim 3, wherein the first array of photonic components is disposed on a first surface of the flexible membrane.

5. The intraocular pressure sensing system of claim 3, wherein the flexible membrane comprises a silicon nitride membrane.

6. The intraocular pressure sensing system of claim 1, further comprising a raised portion located within the chamber on a surface of the second wall.

7. The intraocular pressure sensing system of claim 6, wherein the first array of photonic components is disposed on a surface of the raised portion.

8. The intraocular pressure sensing system of claim 7, further comprising a second array of photonic components disposed on a first surface of the first wall.

9. The intraocular pressure sensing system of claim 7, further comprising an air gap between the first surface of the first wall and the surface of the raised portion.

10. The intraocular pressure sensing system of claim 9, wherein the air gap has a thickness range of 5-10 μm.

11. The intraocular pressure sensing system of claim 1, further comprising a raised section along a perimeter of the first wall and located on a second surface of the first wall, the second surface being opposed and parallel to the first surface of the first wall.

12. The intraocular pressure sensing system of claim 11, further comprising a second layer of anti-reflection coating on a surface of the raised section that is parallel to the first wall.

13. The intraocular pressure sensing system of claim 1, wherein the first array of photonic components comprises photonic components with a hemispherical shape.

14. The intraocular pressure sensing system of claim 13, wherein each photonic component has a diameter between 100-1000 nanometers and a component-to-component pitch between 500-1500 nanometers.

15. The intraocular pressure sensing system of claim 1, wherein the first array of photonic components comprises a 200 μm×200 μm array of photonic components.

16. The intraocular pressure sensing system of claim 2, wherein the plurality of instructions, when executed, cause the processor to determine the intraocular pressure by:
application of a denoising and low pass filter to the resonance spectrum received from the optical spectrometer;
detection of a variation in the resonance spectrum; and
extraction of a pressure reading from the detected variation with a neural network algorithm, wherein the neural network algorithm comprises a feed forward network.

17-20. (canceled)

21. The intraocular pressure sensing system of claim 1, wherein the anti-reflection layer is located between the first wall and the at least one sidewall.

22. The intraocular pressure sensing system of claim 1, wherein the anti-reflection layer is located on a surface of the second wall parallel to the first wall.

23. The intraocular pressure sensing system of claim 1, wherein the anti-reflection layer is located on a side surface of the raised portion, the side surface being perpendicular to the second wall.

24. (canceled)

25. The intraocular pressure sensing system of claim 1, further comprising:
apparatus having a light source configured to shine light into the chamber;
an optical spectrometer configured to receive light reflected from the first array of photonic components inside of the chamber and to output a signal indicative of a resonance spectrum upon receiving the reflected light;
a processor coupled to the optical spectrometer; and
non-transitory memory on which is stored a plurality of instructions that, when executed, cause the processor to:
send the output signal indicative of the resonance spectrum to a remote server for intraocular pressure analysis;
receive an intraocular pressure reading in response to the sending the output signal; and
displaying the intraocular pressure reading on a display area of the apparatus.

26-38. (canceled)
